(19) United States
(12) Patent Application Publication
(10) Pub. No.: US 2014/0010711 A1
(43) Pub. Date: Jan. 9, 2014

(54) Stethoscope Sanitization Apparatuses, Kits, and Methods

(71) Applicant: Tuan Dam, Round Rock, TX (US)

(72) Inventor: Tuan Dam, Round Rock, TX (US)

(21) Appl. No.: 13/862,014

(22) Filed: Apr. 12, 2013

Related U.S. Application Data
(63) Continuation of application No. PCT/US2013/030716, filed on Mar. 13, 2013.
(60) Provisional application No. 61/610,302, filed on Mar. 13, 2012.

(51) Int. Cl. A61L 2/18

(52) U.S. Cl. CPC A61L 2/18
USPC 422/28; 422/108; 422/292

(57) ABSTRACT

Sanitization apparatuses, kits, and methods for sanitizing stethoscopes (e.g., at least one side of a bell and/or diaphragm of a stethoscope chestpiece).

### CROSS-REFERENCE TO RELATED APPLICATIONS

This application is a continuation of co-pending International Patent Application No. PCT/US2013/030716, filed Mar. 13, 2013, which claims priority to U.S. Provisional Patent Application No. 61/610,302, filed Mar. 13, 2012, both of which are incorporated by reference in their entireties.

### BACKGROUND

1. Field of the Invention

The present invention relates generally to sanitization, and, more particularly, but not by way of limitation, to apparatuses, kits, and methods for sanitization of stethoscopes.

2. Description of Related Art

A number of devices and methods for sanitizing objects are known in the art. Bacteria, germs, viruses, and the like can be harmful and/or dangerous (e.g., may transmit colds, the flu, and/or other illnesses). While hand sanitizing gels are known in the art, even if used frequently, they may be relatively ineffective since even recently sanitized hands often grasp objects that have not been sanitized.

### SUMMARY

The present disclosure includes various embodiments of apparatuses, kits, and methods for sanitizing (e.g., disinfecting) stethoscopes.

Some embodiments of the present apparatuses (e.g., for sterilizing a portion of a stethoscope having a bell with a diaphragm) comprise: a housing having a chamber and an opening, the chamber configured to receive a fluid-permeable applicator such that at least a portion of the bell can extend through the opening with the diaphragm disposed in contact with the applicator. Some embodiments further comprise: a fluid-permeable applicator configured to be received in the chamber. In some embodiments, the fluid-permeable applicator is disposed in the chamber. In some embodiments, the fluid-permeable applicator comprises a sponge. Some embodiments further comprise: a sanitization fluid absorbed in the fluid-permeable applicator. In some embodiments, the sanitization fluid is a liquid. In some embodiments, the sanitization fluid is a gel. In some embodiments, the sanitization fluid comprises one or more ingredients selected from the group consisting of: alcohols, ethanol, isopropanol, aldehydes, oxidizing agents, acids, phenolics, ammoniums, and chlorine.

In some embodiments of the present apparatuses, the housing has a peripheral sidewall extending around the chamber, a rear wall extending across a rear side of the chamber between the peripheral sidewall, and a front wall extending across a front side of the chamber between the peripheral sidewall such that the opening is defined between the front wall and the peripheral sidewall. In some embodiments, the peripheral sidewall has a front edge and the front wall is spaced apart from the front edge of the peripheral sidewall. In some embodiments, the chamber is substantially circular.

In some embodiments of the present apparatuses, the applicator has a circular cylindrical shape. In some embodiments, the front wall is configured to be removably coupled to the peripheral sidewall. In some embodiments, the apparatus is configured to sterilize a stethoscope with a dual-sided chestpiece that includes two bells and diaphragm, and where the chamber is configured to receive a fluid-permeable applicator such that a dual-sided chestpiece can extend through the opening with the diaphragm disposed in contact with the applicator. In some embodiments, the front wall is movably coupled to the sidewall such that at least one distance between the rear wall and the front wall is variable. In some embodiments, the front wall is movably coupled to the peripheral sidewall by at least one of: a resilient hinge and a resilient expansion joint. In some embodiments, a portion of the housing outside the chamber is configured to be removably coupled to a portion of a stethoscope other than the bell. In some embodiments, the housing includes one or more projections extending from the peripheral sidewall in a direction away from the chamber, the one or more projections configured to receive the eartips of a stethoscope.

Some embodiments of the present apparatuses further comprise: a liquid crystal display (LCD) screen coupled to the housing. In some embodiments, the LCD screen is configured to display the time. Some embodiments further comprise: an input; an indicator; and a controller coupled to the input and the indicator, the controller configured to receive a signal from the input and cause the indicator to indicate when a predetermined period of time has elapsed after the controller receives a signal from the input. In some embodiments, the indicator comprises a light-emitting diode (LED).

Some embodiments of the present methods comprise: inserting at least a portion of the bell of a stethoscope in the chamber of one of any of the present apparatuses such that a diaphragm (and/or a bell) of the stethoscope contacts the applicator.

Some embodiments of the present refills comprise: a fluid-permeable applicator having a circular-cylindrical shape; a sanitization fluid absorbed in the fluid-permeable applicator; and a package within which the fluid-permeable applicator is sealed.

Some embodiments of the present kits comprise a housing of any of the present applicators; and two or more of the present refills; where the fluid-permeable applicator of each refill is configured to be disposed in the chamber of the housing.

Any embodiment of any of the present apparatuses, kits, refills, or methods can consist of or consist essentially of—rather than comprise/include/contain/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

The feature or features of one embodiment may be applied to other embodiments, even though not described or illustrated, unless expressly prohibited by this disclosure or the nature of the embodiments.

Details associated with the embodiments described above and others are presented below.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers.

FIGS. 1A-1D depict various views of an embodiment of the present stethoscope-sterilization apparatuses.

FIGS. 2A-2C depict various views of a second embodiment of the present stethoscope sterilization apparatuses.

FIGS. 3A-3B depict various views a third embodiment of the present stethoscope sterilization apparatuses.

FIGS. 4A-4B depicts various views of a fourth embodiment of the present stethoscope sterilization apparatuses.

FIGS. 5A-5D depicts various views of a fifth embodiment of the present stethoscope sterilization apparatuses.

FIGS. 6A-6D depict various views of a sixth embodiment of the present stethoscope sterilization apparatuses.

FIGS. 7A-7C depict various views of a seventh embodiment of the present stethoscope sterilization apparatuses.

FIG. 8A-8C depict various views of an eighth embodiment of the present stethoscope-sterilization apparatuses.

FIG. 9A-9B depict various views of a ninth embodiment of the present stethoscope-sterilization apparatuses.

### DESCRIPTION OF ILLUSTRATIVE EMBODIMENTS

The term “coupled” is defined as connected, although not necessarily directly, and not necessarily mechanically; two items that are “coupled” may be unitary with each other. The terms “a” and “an” are defined as one or more unless this disclosure explicitly requires otherwise. The term “substantially” is defined as largely but not necessarily wholly what is specified (and includes what is specified; e.g., substantially 90 degrees includes 90 degrees and substantially parallel includes parallel), as understood by a person of ordinary skill in the art. In any disclosed embodiment, the terms “substantially,” “approximately,” and “about” may be substituted with “within [a percentage] of” what is specified, where the percentage includes 0.1, 1, 5, and 10 percent.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, an apparatus that “comprises,” “has,” “includes” or “contains” one or more elements possesses those one or more elements, but is not limited to possessing only those elements. Likewise, a method that “comprises,” “has,” “includes” or “contains” one or more steps possesses those one or more steps, but is not limited to possessing only those one or more steps.

Further, a device or system that is configured in a certain way is configured in at least that way, but it can also be configured in other ways than those specifically described.

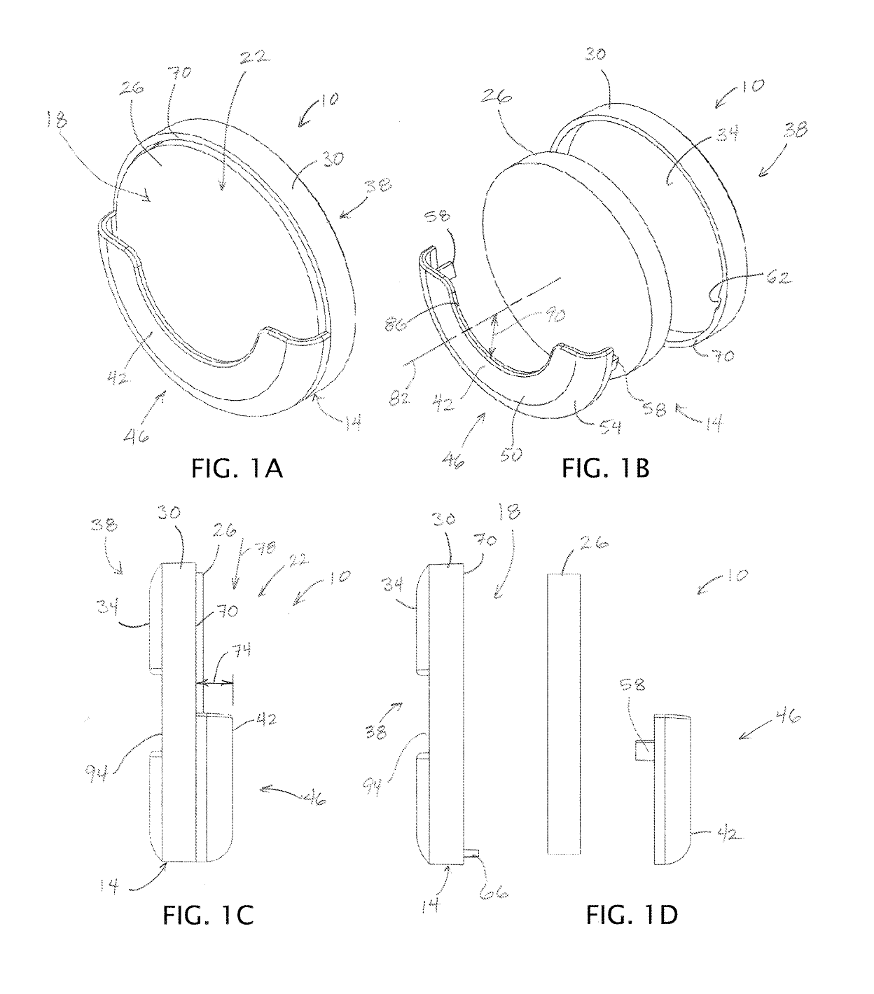

Referring now to the drawings, and more particularly to FIGS. 1A-1D, shown therein and designated by the reference numeral 10 is one embodiment of the present stethoscope sterilization apparatuses. More particularly, FIG. 1A depicts a front perspective view of apparatus 10; FIG. 1B depicts an exploded front perspective view of apparatus 10; FIG. 1C depicts a side view of apparatus 10; and FIG. 1D depicts an exploded side view of apparatus 10. In the embodiment shown, apparatus 10 is configured to hold and/or sterilize a stethoscope, as described in more detail below. In the embodiment shown, apparatus 10 comprises a housing 14 having a chamber 18 and an opening 22. In this embodiment, chamber 18 is configured to receive a fluid-permeable applicator 26 such that at least a portion of a bell of a stethoscope can extend through opening 22 with the diaphragm (and/or bell) of the stethoscope disposed in contact with applicator 26.

In the embodiment shown, housing 14 comprises: a peripheral sidewall 30 extending around chamber 18 (e.g., all of chamber 18, as shown); a rear wall 34 extending across a rear side 38 of chamber 18 between the peripheral sidewall 30; and a front wall 42 extending across a front side 46 of chamber 18 between peripheral sidewall 30 such that opening 22 is defined between front wall 42 and peripheral sidewall 30, as shown. In some embodiments, chamber 18 is substantially circular. For example, in the embodiment shown, peripheral sidewall has a circular cylindrical shape that defines a closed loop around chamber 18. In this embodiment, rear wall 34 is substantially circular and is unitary with peripheral sidewall 30 (i.e., rear wall 34 and sidewall 30 comprise a single piece of material).

In some embodiments, front wall 42 is configured to be removably coupled to peripheral sidewall 30. For example, in the embodiment shown, housing 14 comprises a front member 50 having a curved transition portion 54 and front wall 42. In this embodiment, front member 50 includes a plurality of tabs 58 extending from transition portion 54, with tabs 58 configured to engage corresponding ledges and/or depressions 62 in sidewall 30; and sidewall 30 includes a tab 66 configured to engage corresponding ledges and/or depressions (not shown) in front member 50. In the embodiment shown, peripheral sidewall 30 includes a front edge 70 and front member 50 is configured such that front wall 42 is spaced apart from front edge 70 (e.g., by a distance 74) when front member 50 is coupled to sidewall 30. In some embodiments, front member 50 is interchangeable to accommodate stethoscopes of different sizes. For example, some embodiments of the present kits comprise apparatus 10 with a first front member 50, and one or more additional front members 50 having different sizes such that an end user can select the front member with the size best suited for a particular stethoscope.

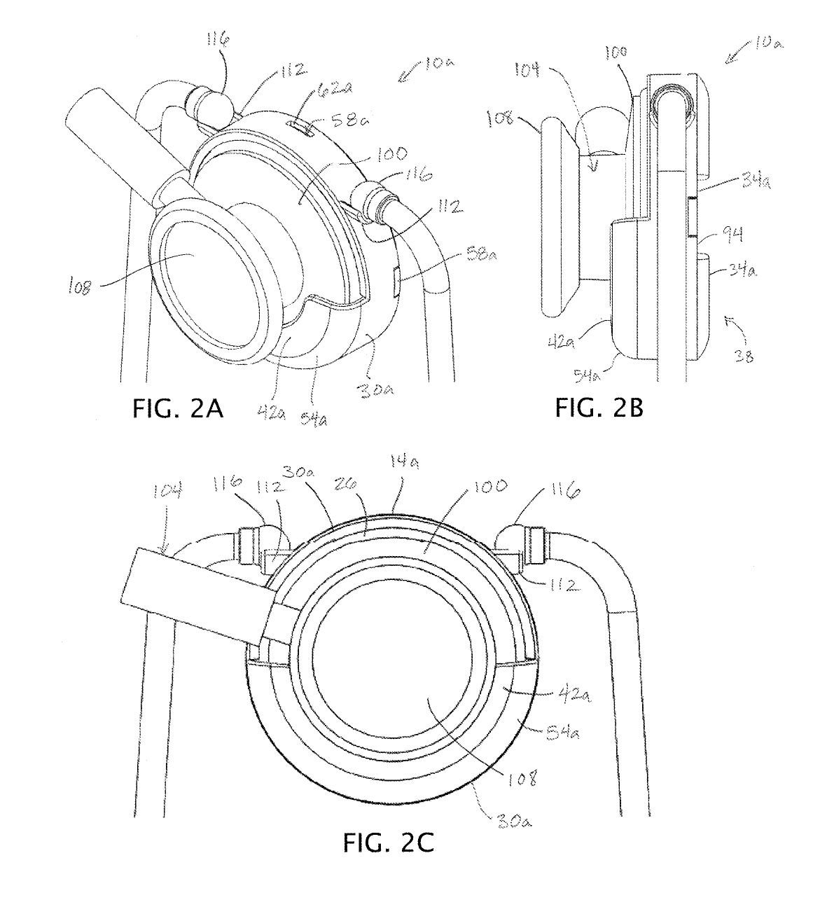

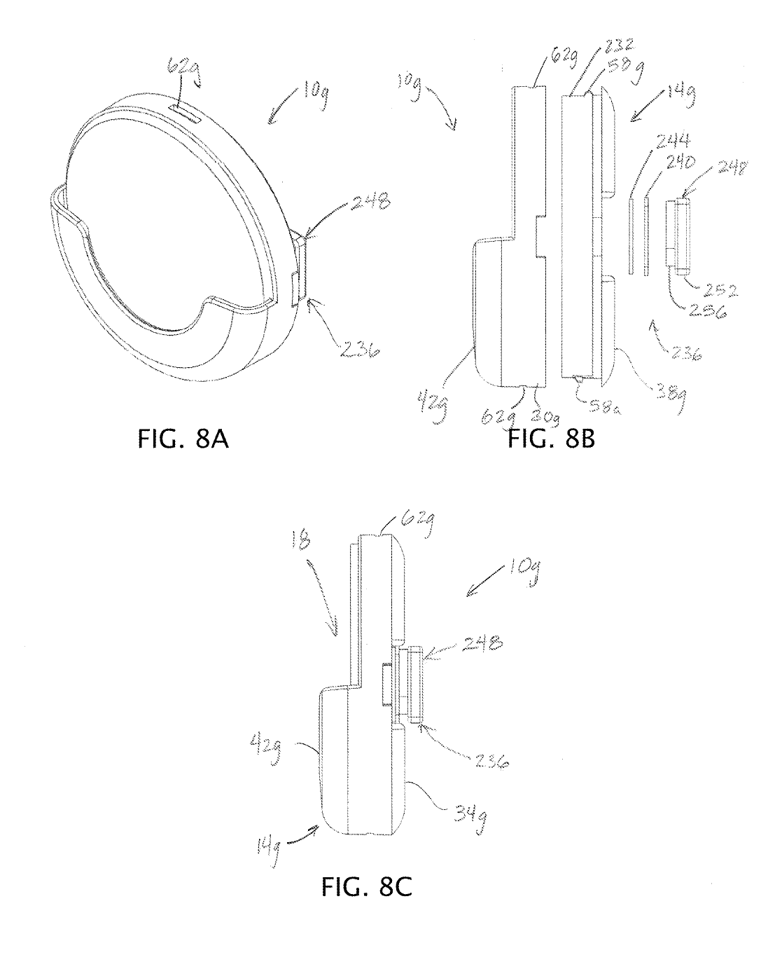

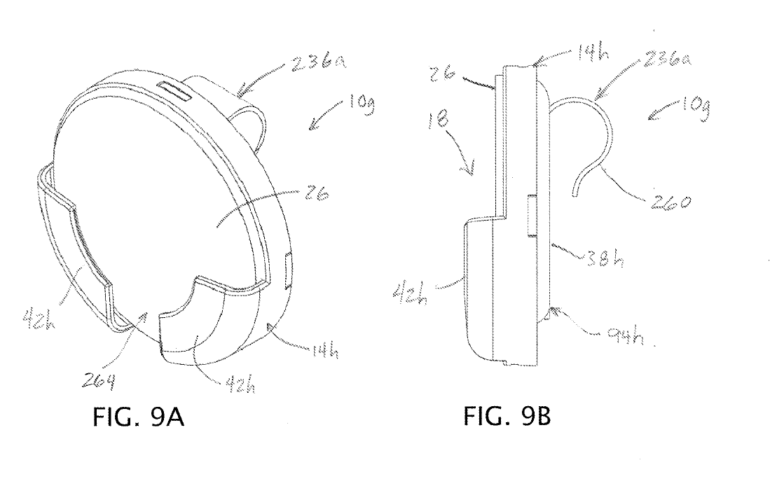

In the embodiment shown, apparatus 10 is configured to permit the bell of a stethoscope to be slid in direction 78 into chamber 18 with the bell (and/or diaphragm) of the stethoscope facing applicator 26. In this embodiment, front wall 42 includes an curved portion extending inward from transition portion 54 toward an axis 82 passing through the radial center of chamber 18, as shown, such that front wall 42 terminates an a curved inner edge 86 that is spaced apart from axis 82 by a radial distance 90 to permit a portion of the chestpiece of the stethoscope to extend out of chamber 18 (e.g., as shown in FIGS. 2A-2C). In the embodiment shown, rear wall 34 of housing 14 includes a laterally-extending recess 94 into which a clip or other supporting structure (not shown) can be coupled to housing, such as, for example, to enable clipping or supporting housing 14 relative to a user's clothing (e.g., shirt, pocket, and/or the like). For example, such a clip or supporting structure can comprise a metal clip, a magnetic clip, or the like (e.g., as depicted in FIGS. 8A-8C and 9A-9B). In other embodiments, the clip or supporting structure can be coupled to the rear wall 34 without recess 108.

In the embodiment shown, fluid-permeable applicator 26 comprises a sponge. In other embodiments, applicator 26 can comprise any suitable material (e.g., felt, fabric, paper, and/or the like) within which a sanitization fluid can be absorbed or otherwise disposed. In the embodiment shown, a sanitization fluid (e.g., liquid, gel, or the like) is absorbed in applicator 26. The sanitization fluid can comprise, for example, one or more ingredients selected from the group consisting of: alcohols, ethanol, isopropanol, aldehydes, oxidizing agents, acids, phenolics, ammoniums, and chlorine. For example, in some embodiments, the sanitization fluid comprises 0.13-percent or 0.26-percent benalkonium chloride (BZK) solution. In some embodiments, the sanitization solution and/or applicator 26 comprises one or more moisture-sensitive desiccants and/or moisture-sensitive chemicals that are configured to change color as applicator 26 dries (e.g., as the sanitization fluid evaporates) to alert a user that applicator 26 should be replaced or re-saturated with sanitization fluid. Examples of moisture-sensitive chemicals are known to include cobalt chloride (CoCl₂) and copper chloride (CuCl₂), such as may be used with humidity indicator cards (HICs) and to indicate saturation levels in desiccants. For example, desiccant beads with a moisture-sensitive chemical can be embedded in applicator 26 (e.g., a rear surface of applicator 26), a moisture-sensitive chemical can be embedded directly in the sponge, or one or more saturation color cards can be embedded in or on the applicator, to change the background or the color of the applicator itself.

FIGS. 2A-2C depict various views of a second embodiment 10a of the present stethoscope sterilization apparatuses. Apparatus 10a is substantially similar in some respects to apparatus 10, and therefore the differences between apparatus 10a and apparatus 10 are primarily described here. As described above for apparatus 10, chamber 18 of apparatus 10a is also configured to receive a fluid-permeable applicator 26 such that at least a portion of a bell 100 of a stethoscope chestpiece 104 can extend through opening 22 with the diaphragm (and/or bell 100 or 108) of the stethoscope disposed in contact with applicator 26. However, apparatus 10a differs from apparatus 10 in that a portion of housing 14a outside chamber 18 is configured to be removably coupled to a portion of the stethoscope other than the bell. Some embodiments can include one or more projections extending from the peripheral sidewall in a direction away from the chamber, with the one or more projections being configured to receive the eartips of a stethoscope. For example, in the embodiment shown, apparatus 10a includes projections 112 configured to receive eartips 116 of stethoscope 104, as shown. Apparatus 10a also differs from apparatus 10 in that apparatus 10a includes a peripheral sidewall 30a that is unitary with transition portion 54a and front wall 42a (i.e., peripheral sidewall 30a, transition portion 54a, and front wall 42a comprise a single piece of material), and a rear wall 34a that is configured to be removably coupled to peripheral sidewall 30a via clips 58a extending from rear wall 34a and corresponding recesses or holes 62a formed in peripheral sidewall 30a.

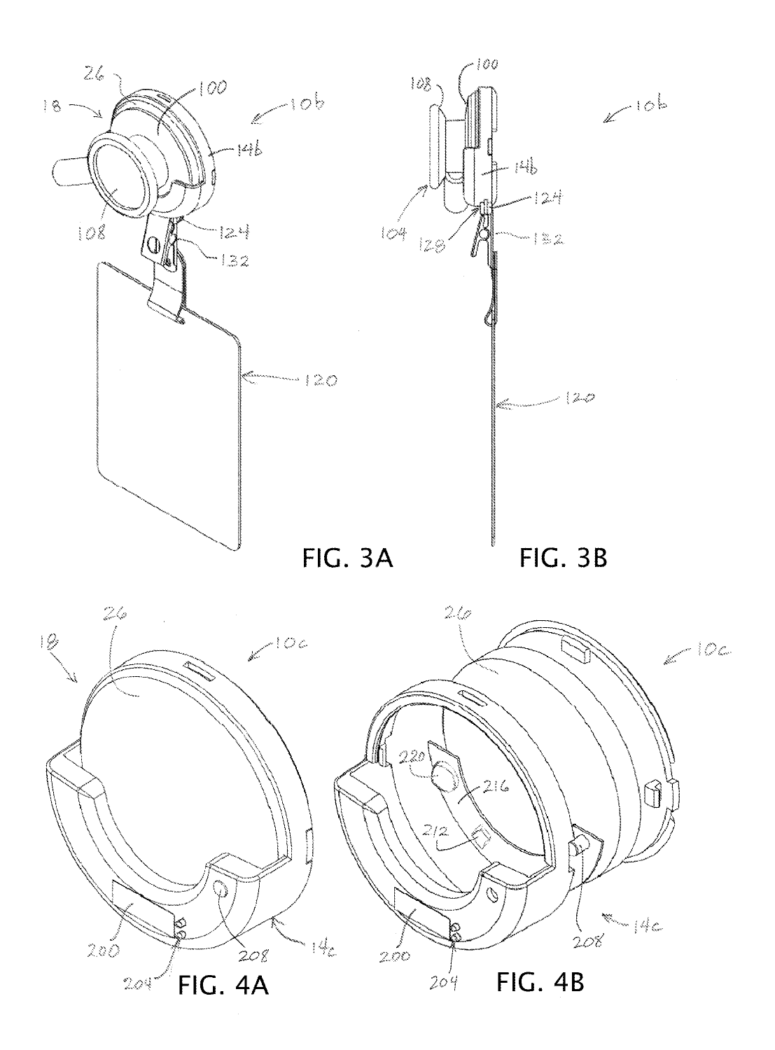

FIGS. 3A-3B depict a third embodiment 10b of the present stethoscope sterilization apparatuses. Apparatus 10b is substantially similar in some respects to apparatus 10a, and therefore the differences between apparatus 10b and apparatus 10a are primarily described here. As described above for apparatus 10a, chamber 18 of apparatus 10b is also configured to receive a fluid-permeable applicator 26 such that at least a portion of a bell 100 of a stethoscope chestpiece 104 can extend through opening 22 with the diaphragm (and/or bell 100 or 108) of the stethoscope disposed in contact with applicator 26. However, apparatus 10b differs from apparatus 10a in that housing 14b does not include projections 112, and housing 14b is instead configured to be coupled to a nametag 120. For example, in the embodiment shown, housing 14b includes a projection 124 extending downward from the bottom of housing 14b and including a recess or opening 128 into which a clip 132 of nametag 120 can be secured to suspend nametag 120 from housing 14b, as shown.

FIGS. 4A-4B depicts various views of a fourth embodiment 10c of the present stethoscope sterilization apparatuses. Apparatus 10c is substantially similar in some respects to apparatus 10b, and therefore the differences between apparatus 10c and apparatus 10b are primarily described here. As described above for apparatus 10b, chamber 18 of apparatus 10c is also configured to receive a fluid-permeable applicator 26 such that at least a portion of a bell 100 of a stethoscope chestpiece 104 can extend through opening 22 with the diaphragm (and/or bell 100 or 108) of the stethoscope disposed in contact with applicator 26. However, apparatus 10c differs from apparatus 10b in that apparatus 10c comprises a liquid crystal display (LCD) screen 200 (e.g., that is configured to display the time) coupled to housing 14c; an input 204; an indicator 208; and a controller 212 (e.g., comprising a clock) coupled to input 204 and indicator 208. In the embodiment shown, controller 212 is coupled to (e.g., included in) a printed circuit board (PCB) 216. In the embodiment shown, apparatus 10c is configured to receive (e.g., coupled to controller 212 such as, for example, via PCB 216) a battery 220, as shown, such that the battery can provide power to permit operation of the apparatus (e.g., controller 212 and indicator 208). Examples of batteries that are suitable for at least some of the present apparatuses includes CR-2016 and CR-2032 lithium batteries.

In the embodiment shown, input 204 comprises a button extending through housing 14c such that the button can be pressed or otherwise activated by a user. In other embodiments, input 204 can comprise a button or other input facing into chamber 18 such that the button or other input is configured to be pressed or otherwise activated when applicator 26 is first disposed in chamber 18. In the embodiment shown, indicator 208 comprise a light-emitting diode (LED). In other embodiments, indicator 208 can comprise any suitable audio and/or visual indicators. In some embodiments, controller 212 is configured to receive a signal from input 204 and cause indicator 208 (e.g., send a signal to indicator to cause indicator 208 to illuminate) to indicate when a predetermined period of time has elapsed after the controller receives a signal from the input. For example, in some embodiments, controller 212 is configured begin a timer (counting either up or down) for a pre-determined period of time (e.g., 12 hours, 24 hours, or more) when indicator 204 is pressed (e.g., corresponding to replacement of applicator 26) and activate indicator 208 once the predetermined period of time has elapsed to indicate to a user when applicator 26 should be replaced again. By way of another example, controller 212 can be coupled (e.g., via PCB 216) to a humidity sensor inside the housing to indicate when low humidity indicates that fluid-permeable applicator may need refilling or replacement.

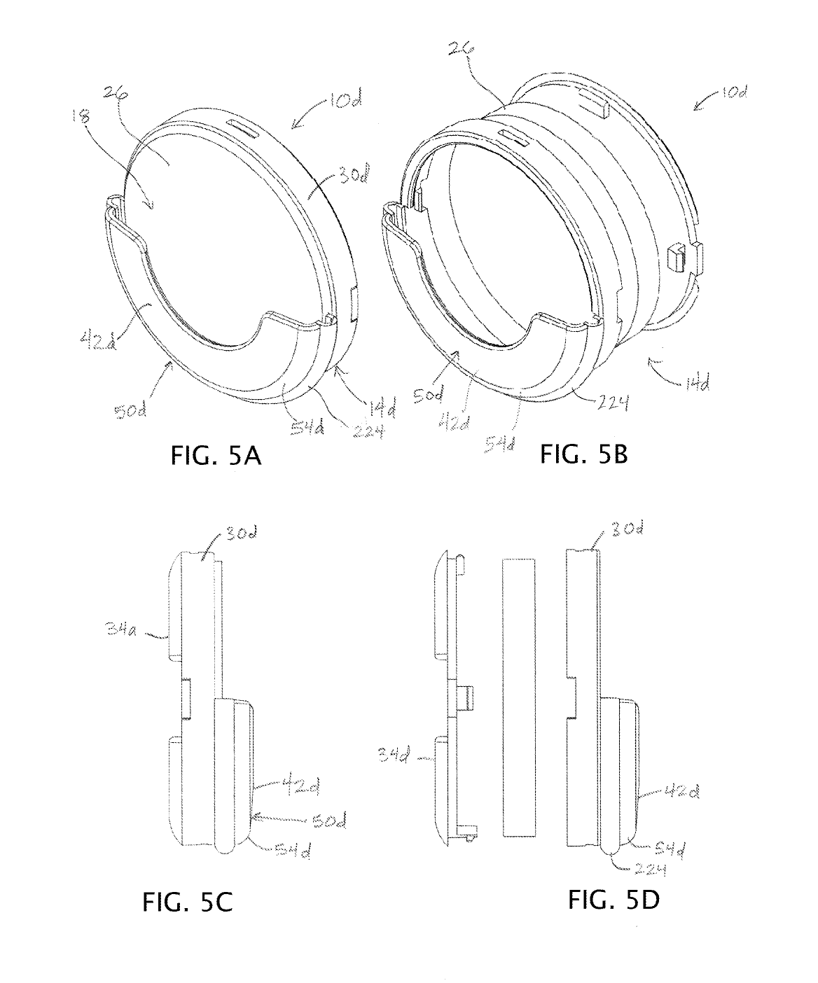

FIGS. 5A-5D depicts various views of a fifth embodiment 10d of the present stethoscope sterilization apparatuses. Apparatus 10d is substantially similar in some respects to apparatus 10a, and therefore the differences between apparatus 10d and apparatus 10a are primarily described here. As described above for apparatus 10a, chamber 18 of apparatus 10d is also configured to receive a fluid-permeable applicator 26 such that at least a portion of a bell 100 of a stethoscope chestpiece 104 can extend through opening 22 with the diaphragm (and/or bell 100 or 108) of the stethoscope disposed in contact with applicator 26. However, apparatus 10d differs from apparatus 10a in that front member 50d (including front wall 42d and transition portion 54d) is movably coupled to peripheral sidewall 30d such that at least one distance between front wall 42d and rear wall 34d is variable. For example, in the embodiment shown, front member 50d is movably coupled to peripheral sidewall 30 by a flexible and resilient (e.g., expansion) joint 224 along the perimeter of transition portion 54d, as shown. Joint 224 can comprise any suitable material (e.g., polymer, natural or synthetic rubber, and/or the like) and can have any suitable shape. For example, in the embodiment shown, joint 224 has a U-shaped cross-section such that front member 50a is movable relative to sidewall 30d, and joint 224 comprises a resilient polymer such that if the joint is stretched relative to a neutral or relaxed state (e.g., if a bell 100 is disposed in chamber 18), joint 224 will provide a biasing force on front wall 42d in the direction of rear wall 34d (e.g., tending to hold the bell in the chamber and press the stethoscope bell and/or diaphragm into applicator 26). In this embodiment, joint 224 extends along substantially the entire perimeter of transition portion 54d such that front wall 42d can (but need not) be maintained in a plane that is substantially parallel to a plane in which rear wall 34d is disposed, as front wall 42d is moved relative to rear wall 34d. In some embodiments, joint 224 is configured to permit the distance between front wall 42d and rear wall 34d to vary by between 0.0125 and 0.25 inches (e.g., equal to, or between any two of: 0.125, 0.2, 0.25 inches).

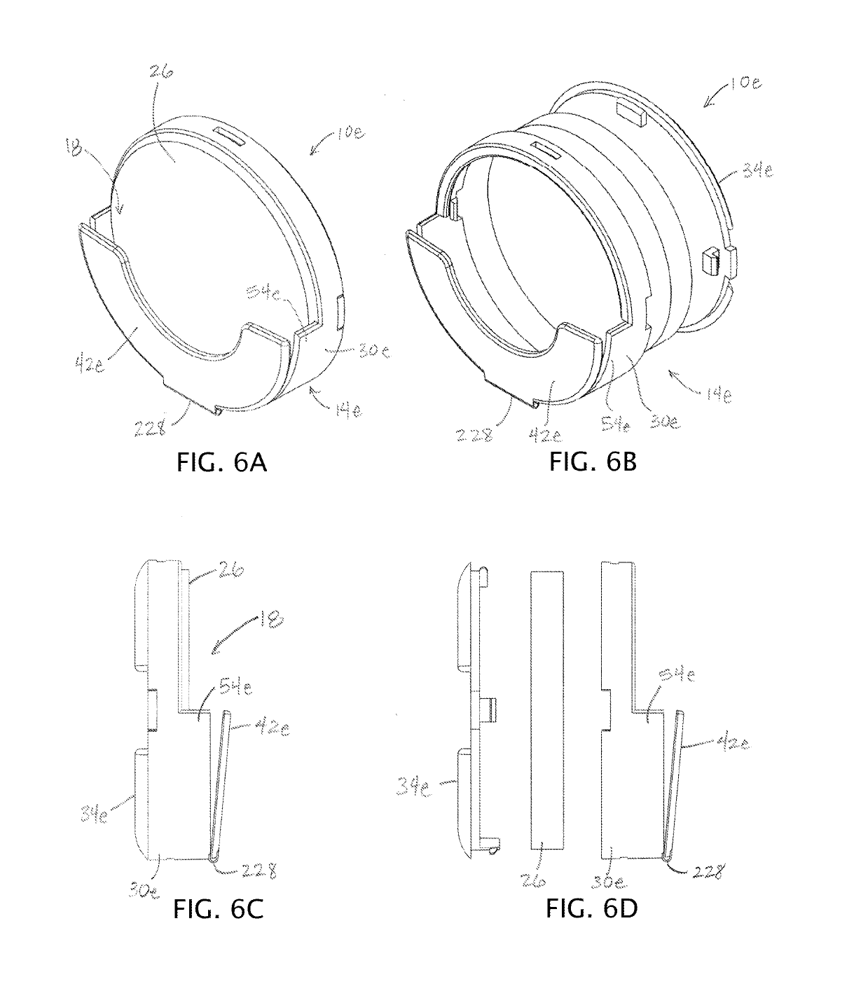

FIGS. 6A-6D depict various views of a sixth embodiment 10e of the present stethoscope sterilization apparatuses. Apparatus 10e is substantially similar in some respects to apparatus 10d, and therefore the differences between apparatus 10e and apparatus 10d are primarily described here. As described above for apparatus 10d, chamber 18 of apparatus 10e is also configured to receive a fluid-permeable applicator 26 such that at least a portion of a bell 100 of a stethoscope chestpiece 104 can extend through opening 22 with the diaphragm (and/or bell 100 or 108) of the stethoscope disposed in contact with applicator 26. However, apparatus 10e differs from apparatus 10d in that and transition portion 54e is unitary with peripheral sidewall 30e and front wall 42e is movably coupled to transition portion 54e by a resilient hinge 228 such that at least one distance between front wall 42e and rear wall 34e is variable. In the embodiment shown, hinge 228 is a “living” hinge that is unitary with at least one of front wall 34a and transition portion 54e. As described above for joint 224, hinge 228 comprises a resilient polymer such that if the hinge is stretched relative to a neutral or relaxed state (e.g., if a bell 100 is disposed in chamber 18), hinge 228 will provide a biasing force on front wall 42e in the direction of rear wall 34e (e.g., tending to hold the bell in the chamber and press the stethoscope bell and/or diaphragm into applicator 26).

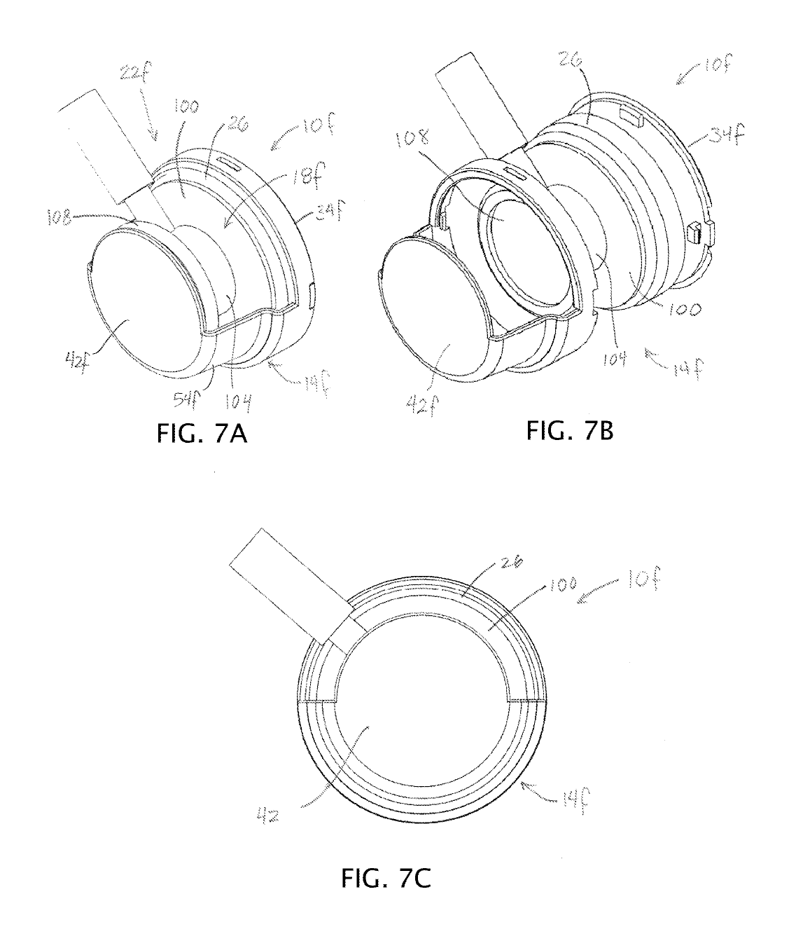

FIGS. 7A-7C depict various views of a seventh embodiment 10f of the present stethoscope sterilization apparatuses. Apparatus 10f is substantially similar in some respects to apparatus 10a, and therefore the differences between apparatus 10f and apparatus 10a are primarily described here. As described above for apparatus 10a, chamber 18f of apparatus 10f is also configured to receive a fluid-permeable applicator 26 such that at least a portion of a bell 100 of a stethoscope chestpiece 104 can extend through opening 22 with the diaphragm (and/or bell 100 or 108) of the stethoscope disposed in contact with applicator 26. However, apparatus 10f differs from apparatus 10a in that housing 14f defines chamber 18f that is configured to receive a fluid-permeable applicator such that a dual-sided chestpiece 104 can extend through opening 22f with the diaphragm disposed in contact with applicator 26. In this embodiment, and as shown in FIGS. 6A and 6C, chamber 18f and opening 22f are configured to receive both bells 100 and 108 of dual-sided chestpiece 104 such that both bells 100 and 108 are disposed between front wall 42f and rear wall 34f. For example, in the embodiment shown, the distance between front wall 42f and rear wall 34f is increased (e.g., relative to a corresponding distance in apparatus 10a) with an extended transition portion 54f. In this embodiment, front wall 42f is substantially circular and is sized to correspond to the size of bell 108 (which is smaller than bell 100 for the stethoscope shown). In other embodiments, front wall 42f is substantially the same size as rear wall 34f such that the stethoscope chestpiece 104 can be disposed in chamber 18f with either of bells 100 and 108 in contact with applicator 26.

FIGS. 8A-8C depict various views of a eighth embodiment 10g of the present stethoscope sterilization apparatuses. Apparatus 10g is substantially similar in some respects to apparatus 10a, and therefore the differences between apparatus 10g and apparatus 10a are primarily described here. As described above for apparatus 10a, chamber 18 of apparatus 10g is also configured to receive a fluid-permeable applicator 26 such that at least a portion of a bell 100 of a stethoscope chestpiece 104 can extend through opening 22 with the diaphragm (and/or bell 100 or 108) of the stethoscope disposed in contact with applicator 26. However, apparatus 10g differs from apparatus 10a in that rear wall 34g comprises a peripheral projection 232 substantially similar in shape, but slightly smaller than, peripheral sidewall 30g such that projection 232 can be inserted into peripheral sidewall 30g with tabs 58g engaging openings 62g. In the embodiment shown, apparatus 10g also comprises a magnetic clip 236 coupled to recess 94g of rear wall 34g. In this embodiment, clip 236 comprises a ferromagnetic (e.g., metallic) strip 240 coupled to rear wall 34g (e.g., by a double-sided adhesive tape 244) and a magnetic retainer 248 that comprises a supporting strip 252 (e.g., comprising a magnetically inert polymer, ferromagnetic metal, and/or any other suitable material) and one or more magnets 256 couples to strip 252. Retainer 248 can, for example, be disposed on the inside of a healthcare provider's shirt and apparatus 10g can be disposed on the outside of the shirt with strip 240 aligned with retainer 248 such that the magnetic interaction between strip 240 and retainer 248 will hold apparatus 10g in substantially fixed relation relative to the shirt. Clip 236 can also be used or included with other embodiments of the present apparatuses (e.g., 10, 10a, 10b, 10c, 10d, 10e, 10f Other embodiments can include any suitable clip or fastening structure (e.g., Velcro, double-sided tape, spring clips, snaps, etc.

FIGS. 9A-9B depict various views of a ninth embodiment 10h of the present stethoscope sterilization apparatuses. Apparatus 10h is substantially similar in some respects to apparatus 10g, and therefore the differences between apparatus 10h and apparatus 10h are primarily described here. As described above for apparatus 10a, chamber 18 of apparatus 10g is also configured to receive a fluid-permeable applicator 26 such that at least a portion of a bell 100 of a stethoscope chestpiece 104 can extend through opening 22 with the diaphragm (and/or bell 100 or 108) of the stethoscope disposed in contact with applicator 26. However, apparatus 10h differs from apparatus 10g in that rear wall 34g comprises a vertical recess 94h and a clip 236a that is configured to be clipped over the edge of a shirt pocket or other horizontal edge or structure. In this embodiment shown, clip 236a is coupled to rear wall 34h in recess 94h, as shown, such that the hook portion 260 can be clipped or hung over a horizontal edge or other structure to support apparatus 10h. Apparatus 10h also differs from apparatus 10g in that front wall 42h of apparatus 10h is divided into two parts on either side of a vertical opening 264.

Some embodiments of the present methods comprise: inserting at least a portion of the bell (e.g., 100 or 108) of a stethoscope (e.g., of chestpiece 104) in the chamber of an one of the present apparatuses (e.g., 10, 10a, 10b, 10c, 10d, 10e, 10f, 10g, 10h) such that a diaphragm of the stethoscope contacts the applicator.

Some embodiments of the present refills comprise: a fluid-permeable applicator 26 (e.g., having a circular-cylindrical shape); a sanitization fluid absorbed in the fluid-permeable applicator; and a package (e.g., a round or rectangular sealed foil pouch, or a bag such as, for example, a round or rectangular vacuum-sealed plastic bag, a rectangular zipper-sealed plastic bag, or the like) within which the fluid-permeable applicator is sealed.

Some embodiments of the present kits comprise a housing of the present apparatuses (e.g., 14, 14a, 14b, 14c, 14d, 14e, 14f, 14g, 14h) and/or one of the present apparatuses; and two or more (e.g., two, three, four, five, six, seven, eight, nine, ten, or more) of the present refills (e.g., each comprising an applicator, within which sanitization fluid is absorbed, that is sealed in a package); where the applicator of each refill apparatus is configured to be (e.g., interchangeably) disposed in the chamber 18 of the housing and/or apparatus.

Any of the present apparatuses, kits, and/or methods can be configured to have any of the features described or depicted for any of the other apparatuses, kits, and/or methods.

The above specification and examples provide a complete description of the structure and use of illustrative embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the present apparatuses, kits, refills, and methods are not intended to be limited to the particular forms disclosed. Rather, they include all modifications and alternatives falling within the scope of the claims, and embodiments other than the one shown may include some or all of the features of the depicted embodiment. For example, components may be omitted or combined as a unitary structure, and/or connections may be substituted. Further, where appropriate, aspects of any of the examples described above may be combined with aspects of any of the other examples described to form further examples having comparable or different properties and addressing the same or different problems. Similarly, it will be understood that the benefits and advantages described above may relate to one embodiment or may relate to several embodiments.

The claims are not intended to include, and should not be interpreted to include, means-plus- or step-plus-function limitations, unless such a limitation is explicitly recited in a given claim using the phrase(s) “means for” or “step for,” respectively.

1. A apparatus for sterilizing a portion of a stethoscope having a bell with a diaphragm, the apparatus comprising:
a housing having a chamber and an opening, the chamber configured to receive a fluid-permeable applicator such that at least a portion of the bell can extend through the opening with the diaphragm disposed in contact with the applicator.

2. The apparatus of claim 1, further comprising:
a fluid-permeable applicator configured to be received in the chamber.

3. The apparatus of claim 2, where the fluid-permeable applicator is disposed in the chamber.

4. The apparatus of claim 3, where the fluid-permeable applicator comprises a sponge.

5. The apparatus of claim 1, further comprising:
a sanitization fluid absorbed in the fluid-permeable applicator.

6. The apparatus of claim 5, where the sanitization fluid is a liquid.

7. The apparatus of claim 6, where the sanitization fluid is a gel.

8. The apparatus of claim 1, where the sanitization fluid comprises one or more ingredients selected from the group consisting of: alcohols, ethanol, isopropanol, aldehydes, oxidizing agents, acids, phenolics, ammoniums, and chlorine.

9. The apparatus of claim 1, where the housing has a peripheral sidewall extending around the chamber, a rear wall extending across a rear side of the chamber between the peripheral sidewall, and a front wall extending across a front side of the chamber between the peripheral sidewall such that the opening is defined between the front wall and the peripheral sidewall.

10. The apparatus of claim 9, where the peripheral sidewall has a front edge and the front wall is spaced apart from the front edge of the peripheral sidewall.

11. The apparatus of claim 9, where the chamber is substantially circular.

12. The apparatus of claim 11, where the applicator has a circular cylindrical shape.

13. The apparatus of claim 1, where the front wall is configured to be removably coupled to the peripheral sidewall.

14. The apparatus of claim 1, where the apparatus is configured to sterilize a stethoscope with a dual-sided chestpiece that includes two bells and diaphragm, and where the chamber is configured to receive a fluid-permeable applicator such that a dual-sided chestpiece can extend through the opening with the diaphragm disposed in contact with the applicator.

15. The apparatus of claim 1, where the front wall is movably coupled to the sidewall such that at least one distance between the rear wall and the front wall is variable.

16. The apparatus of claim 15, where the front wall is movably coupled to the peripheral sidewall by at least one of: a resilient hinge and a resilient expansion joint.

17. The apparatus of claim 1, where a portion of the housing outside the chamber is configured to be removably coupled to a portion of a stethoscope other than the bell.

18. The apparatus of claim 17, where the housing includes one or more projections extending from the peripheral sidewall in a direction away from the chamber, the one or more projections configured to receive the eartips of a stethoscope.

19. The apparatus of claim 1, further comprising:
a liquid crystal display (LCD) screen coupled to the housing.

20. The apparatus of claim 19, where the LCD screen is configured to display the time.

21. The apparatus of claim 1, further comprising:
an input;
an indicator;
a controller coupled to the input and the indicator, the controller configured to receive a signal from the input and cause the indicator to indicate when a predetermined period of time has elapsed after the controller receives a signal from the input.

22. The apparatus of claim 21, where the indicator comprises a light-emitting diode (LED).

23. A method comprising:
inserting at least a portion of the bell of a stethoscope in the chamber of an apparatus of claim 1 such that a diaphragm of the stethoscope contacts the applicator.

24. A refill comprising:
a fluid-permeable applicator having a circular-cylindrical shape;
a sanitization fluid absorbed in the fluid-permeable applicator; and
a package within which the fluid-permeable applicator is sealed.

25. A kit comprising:
a housing of the apparatus of claim 1; and
two or more refills of claim 24;
where the fluid-permeable applicator of each refill apparatus is configured to be disposed in the chamber of the housing.
